(19) United States
(12) Patent Application Publication
(10) Pub. No.: US 2004/0106865 A1
(43) Pub. Date: Jun. 3, 2004

(54) Magnetic resonance apparatus with a movable gradient coil unit

(76) Inventors: Johann Schuster, Oberasbach (DE); Stefan Stocker, Erlangen (DE)

Correspondence Address: SCHIFF HARDIN, LLP, PATENT DEPARTMENT, 6600 SEARS TOWER, CHICAGO, IL, 60606-6473, US

(21) Appl. No.: 10/611,456

(22) Filed: Jul. 1, 2003

(30) Foreign Application Priority Data
Jul. 1, 2002 (DE) 102 29 490.9

(51) Int. Cl. A61B 5/05

(52) U.S. Cl. 600/410

(57) ABSTRACT

A magnetic resonance apparatus for examinations having a local gradient coil unit (insert gradient coils) has a driven support mechanism (patient bed) with which the examination subject can be introduced into the examination space of the magnetic resonance apparatus, and a coupling device with which a gradient coil unit can be coupled to the patient bed and can be moved into the examination space by the driven patient bed. The gradient coil unit is positioned in the examination space by the patient bed and does not required its own drive.

### BACKGROUND OF THE INVENTION

1. Field of the Invention

The present invention is directed to a magnetic resonance apparatus for examinations having a local gradient coil unit (insert gradient coils) as well as to a gradient coil unit suitable for such a magnetic resonance apparatus.

2. Description of the Prior Art

Magnetic resonance systems have been employed for many years in medicine and in biophysics for acquiring images from the inside of the body of an examination subject. Magnetic resonance tomography is based on the physical phenomenon of nuclear magnetic resonance. In the examination procedure, the examination subject is exposed to a strong, constant magnetic field, causing the previously irregularly oriented nuclear spins of the atoms to align in the examination subject. These unordered nuclear spins are excited to a specific oscillations (resonant frequency) by means of radio-frequency waves. This oscillation generates the actual measured signal (RF response signal) for the actual image acquisition in magnetic resonance tomography that is picked up by suitable reception coils.

A pre-condition for the Image acquisition is an exact identification of the respective locations at which the Rf response signals arise in the examination subject (location information, or location encoding). This location information is acquired by means of auxiliary magnetic fields (magnetic gradient fields) that are generated along the three spatial directions by gradient coils. The gradient field superimposed on the basic field is designed such that the field strength, and thus the resonant frequency as well are different in every volum elem nt. When RF energy at a defined resonant frequency is emitted into the subject, only those atoms can be excited that are situated at a location at which the magnetic fields satisfy the corresponding resonance condition. Suitable changes of the gradient fields make it possible to shift the location of such a volume element at which the resonance condition is satisfied in a defined way, and to thus scan the desired region.

For the examination, the subject is introduced into the examination space of the magnetic resonance apparatus and positioned therein. In order to be able to accept examination subjects of different sizes, the examination space must have a minimum size. For medical applications, the size is selected such that a patient can be completely introduced into the examination space.

A local gradient coil unit (insert gradient coil) can be employed for the examination of specific regions of an examination subject, for example the head of a patient. U.S. Pat. No. 5,185,576 discloses such a local gradient coil unit that is introduced into the examination space of the magnetic resonance apparatus.

With their smaller dimensions, local gradient coils have advantages over the built-in whole-body gradient coils of a magnetic resonance apparatus in terms of the obtainable gradient intensity and the power demands made on the gradient amplifier that feeds the gradient coil unit. For operation, the local gradient coil unit together with the integrated, local radio-frequency antenna must be firmly anchored in the examination space of the magnetic resonance apparatus in order to withstand the forces acting on them.

In some embodiments of local gradient coils the securing of the local radio-frequency antenna in the magnetic resonance apparatus and the removal therefrom can require several hours, since the whole-body antenna of the magnetic resonance apparatus must be un-installed and re-installed in some cases.

U.S. Pat. No. 5,311,134 discloses a magnetic resonance apparatus that has a rail-like guide mechanism on which a movable gradient coil unit can be moved. The magnetic resonance apparatus further has a patient bed with which a patient lying thereon can be positioned both in the interior hollow volume of the examination space as well as in the interior hollow volume of the local gradient coil unit.

Manually inserting and mounting the local gradient coils, which weigh up to 250 kg, requires a great outlay on the part o the operating personnel in terms of time; and exertion.

U.S. Pat. No. 5,783,943 discloses a device for inserting and positioning a local gradient coil unit in an examination space of a magnetic resonance apparatus wherein the local gradient coil is coupled to and movable by the driven patient bed.

### SUMMARY OF THE INVENTION

An object of the present invention is to provide a magnetic resonance apparatus and a gradient coil unit for insertion into the magnetic resonance apparatus that enable a simple and easy positioning of the gradient coil unit in the examination space of the magnetic resonance apparatus.

This object is achieved in accordance with the invention in a magnetic resonance apparatus having a driven patient bed mechanism with which the examination subject can be introduced into the examination space of the magnetic resonance apparatus having a coupling device with which a gradient coil unit can be coupled to the supporting mechanism and with which the supporting mechanism (patient bed) is movable in the examination space. The coupling device automatically firmly joins the gradient coil unit to the patient bed mechanism when the patient bed bearing mechanism moves toward the local gradient coil unit from one direction and releases as soon as the patient bed bearing mechanism again moves in that direction. The gradient coil unit is positioned in the examination space by the patient bed and does not require its own drive. Locking of the gradient coil unit in the examination space during operation can likewise ensue with the patient bed and/or by means of a lock mechanism attached in the examination space that fixes the gradient coil unit as soon as the it has been moved into a specific position by the patient bed. The patient bed and its drive are appropriately designed for this purpose.

A cart for the gradient coil unit is attachable at one of the openings of the examination space for easy withdrawal and insertion of the gradient coil unit and the assembly of the needed connections. The gradient coil unit can be moved out of the examination space onto the cart by the patient bed and can be dismantled there in an easily accessible way. This enables a fast change between various examination modes and shortens the refitting times.

The cart can be pivotably attachable to the magnetic resonance apparatus, then the patient bed can be moved out of the examination space past the gradient coil unit that has been pivoted away from the opening of the examination space. For a renewed insertion of the gradient coil unit disposed on the cart, the cart is again pivoted against the opening of the examination space.

In an embodiment, the connections needed for the gradient coil unit are integrated in the patient bed. This enhances the reliability and shortens the time needed for the mounting of the gradient coil unit.

A glide and guide device at the cart and in the examination space for the gradient coil unit allow a stable mounting of the patient bed and its drive.

The gradient coil unit is rigidly but releasably connected to the patient bed by the coupling device. An automatic or remote controllable coupling device enables exact positioning by means of the controller of the magnetic resonance apparatus and an easy insertion of the gradient coil unit into and withdrawal af the gradient coil unit out of the examination space.

Existing systems can be easily remodeled according to the present invention by suitable modifications at the patient bed and/or gradient coil unit.

### DESCRIPTION OF THE DRAWINGS

FIG. 1 is a side view, partly in section, of an exemplary embodiment of the inventive magnetic resonance apparatus with an Inserted gradient coil unit.

FIG. 1A is a vertical longitudinal section through the exemplary embodiment of the inventive magnetic resonance apparatus of FIG. 1.

FIG. 2 shows the exemplary embodiment of the inventive magnetic resonance apparatus of FIG. 1 with the gradient coil unit withdrawn.

FIG. 3 shows the exemplary embodiment of the inventive magnetic resonance apparatus with the withdrawn gradient coil unit in a horizontal section.

FIG. 4 shows an example of a lock mechanism for locking the gradient coil unit in a specific position in the examination space of the inventive magnetic resonance apparatus.

### DESCRIPTION OF THE PREFERRED EMBODIMENTS

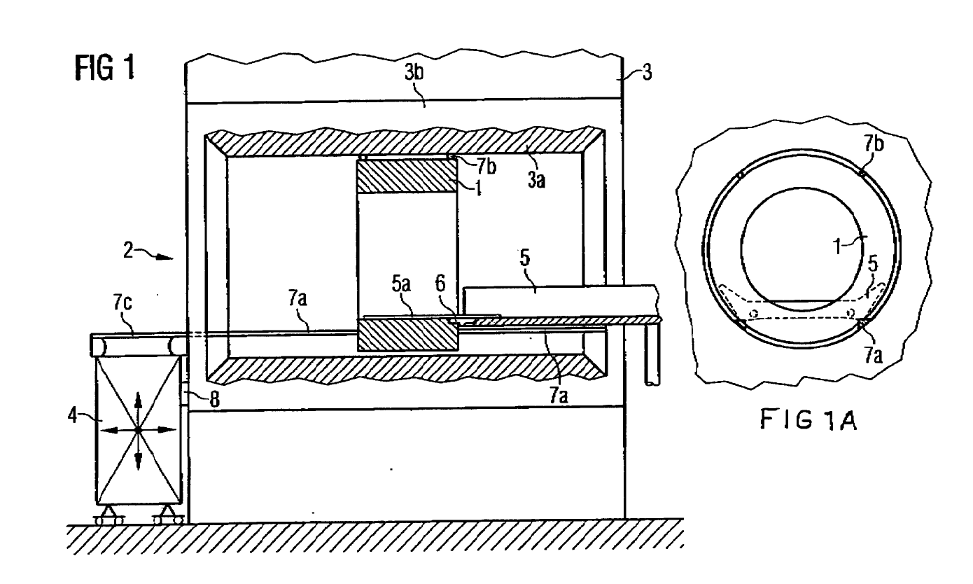

FIGS. 1 and 1A shows a side section and vertical longitudinal section of an exemplary embodiment of the inventive magnetic resonance apparatus. The static basic field magnet 3b and the gradient coil system 3a are basic components of the magnetic resonance apparatus 3, which have a hollow interior examination space 2.

The gradient coil unit 1 has already been introduced in the examination space 2 of the magnetic resonance apparatus 3 shown in FIG. 1 and has been connected to the patient bed 5 via a coupling device 6. The head support 5a of the patient bed extends into the hollow interior of the gradient coil unit 1.

Rails 7a are attached in the lower part of the examination space 2 for guiding the gradient coil unit 1 and the patient bed 5. The centering of the gradient coil unit 1 within the hollow-cylindrical examination space 2 ensues by means or rollers 7b attached in pairs at the upper part pf the gradient coil unit 1.

Guidance and fixing of the gradient coil unit 1 in the examination space 2 can be designed in some other way, for example in conformity with the shape and material of the examination space 2. In the arrangement shown in FIG. 1, the guide device 7a of the patient bed 5 is also used for guiding the gradient coil unit 1.

A movable cart 4 attachable to the left side of the examination space 2 serves for the acceptance and storing of the gradient coil unit 1. A docking device 8 positions the rails 7c attached to the cart 4 relative to the rails 7a of the examination space 2, so that the gradient coil unit 1 can be moved into or removed from the examination space 2 sliding on the rails 7c, 7a.

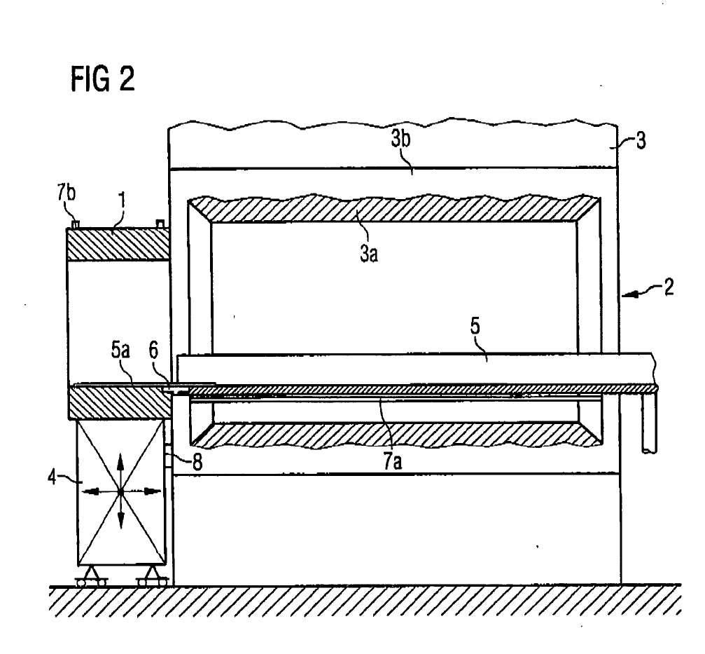

FIG. 2 shows the arrangement of FIG. 1, wherein the gradient coil unit 1 is moved onto the cart 4. For storing the gradient coil unit 1, the gradient coil unit 1 is uncoupled from the patient bed, the patient bed 5 is introduced into the examination space 2, the cart 4 is undocked from the magnetic resonance apparatus 3 and moved to its storage location together with the gradient coil unit 1 placed thereon.

For inserting the gradient coil unit 1 into the examination space 2, th gradient coil unit 1 is fetched from its storage location with the cart 4, and the cart 4 is docked to the opening of the examination space 2. The cart 4 in the arrangement shown in FIG. 2 is height-adjustable and is thus especially adaptable to different forms of magnetic resonance apparatus 3.

The positioning of the rails 7c (FIG. 1) and 7a relative to one another and the coupling event can be automatically triggered after the successful docking. When the rails 7c and 7a are positioned relative to one another, the patient bed 5 can be moved toward the left and the gradient coil unit 1 can be coupled to the patient bed 5. The patient bed 5 subsequently moves the gradient coil unit 1 into the examination space 2 to a predefined position.

The coupling and uncoupling of the gradient coil unit 1 ensues by means of an automatic coupling device 6 that—when the patient bed 5 moves to the left toward the gradient coil unit 1—firmly joins the gradient coil unit 1 and the patient bed 5 to one another and releases the connection when the patient bed 5 is again moved (at least briefly) toward the left (bipolar coupling state modified by compressing the coupling device 6). Additionally, the coupling 6 can be remotely actuated.

After the uncoupling of the gradient coil unit 1 at a predefined position, the patient bed 5 moves toward the right out of the examination space 2 in order to accept the patient. With, for example, the patient's head placed on the head support 4a at the front end of the patient bed 5, the patient is moved into the examination space 2, whereby the head support 5a moves into the gradient coil unit 1 and is positioned therein.

Modifications of the described sequences can be necessary when, for example, the connections needed for the operation of the gradient coil unit 1 are int grated in the pati nt b d and some of these connections cannot be automatically produced when coupling. It is thereby advantageous to have the operating personnel produce the necessary connections in the withdrawn position.

A coupling of the gradient coil unit 1 to the patient bad 5 during operation can likewise be necessary if sufficient fixing means for holding the gradient coil unit 1 are not present in the examination space 2, and the gradient coil unit 1 must be fixed over or via the patient bed 5.

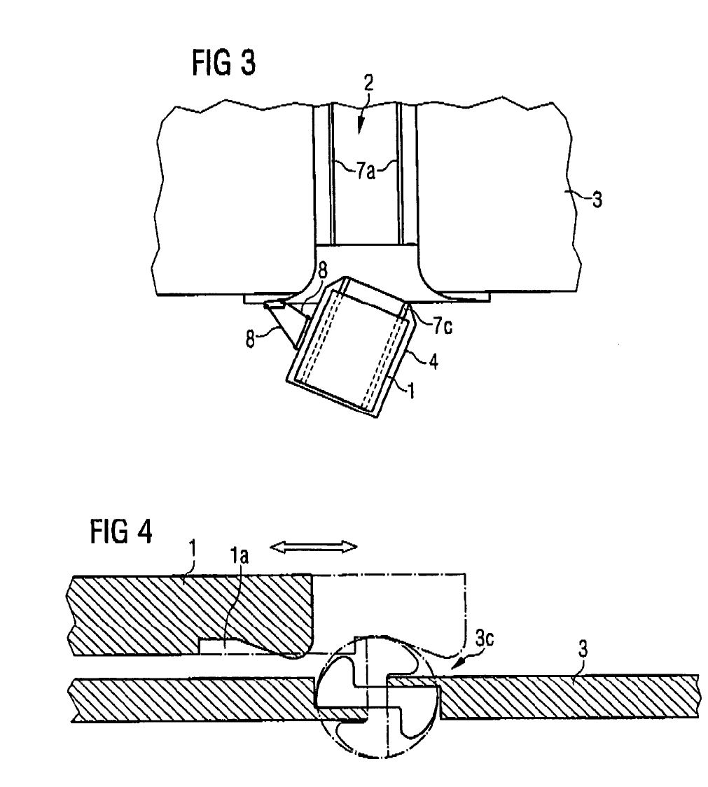

FIG. 3 shows an exemplary embodiment of the invention wherein the cart 4 can be pivoted toward and away from the opening of the examination space 2. In this exemplary embodiment, the deposit device 4 with the gradient coil unit 3 is brought laterally toward the magnetic resonance apparatus 3 either manually or with an electrical drive. As a result of the docking device 8, the insertion device 4 is pivotable such that the rails 7a and 7c are exactly positioned relative to one another in the pivoted-in condition.

The pivotably designed docking device 8 is especially advantageous given a changing operation/use of local gradient coil and the permanently installed whole-body gradient coil, whereby the patient bed 5 must be partly moved out of the opening of the examination space 2 of the magnetic resonance apparatus 3. In this case, the gradient coil unit 2 is moved out of the examination space 2 onto the cart 4 and pivoted toward the side. The patient bed 5 can then be moved past the gradient coil unit 2 in this position. A renewed insertion of the gradient coil unit 1 is possible without another assembly of the necessary connections of the gradient coil unit 1.

The positioning of the gradient coil unit 1 In the examination space 2 ensues by means of software in interaction with the magnetic resonance apparatus 3 or via mechanically adjustable stops,

In an excerpt, FIG. 4 shows an example of th locking of the gradient coil unit 1 in the examination space 2 in a specific inserted position.

The lock mechanism 3c of the magnetic resonance apparatus 3 has two cams. The first cam exhibits a heavier weight than the second cam—for example by being fashioned of a high-density material—so that the position of the lock mechanism 3c shown with solid lines is automatically set when the gradient coil unit 1 is not inserted or not completely inserted. Given an insertion of the gradient coil unit 1 from the left into the examination space 2, the lock mechanism 1a of the gradient coil unit 1 entrains the second cam and places the lock mechanism 3c into a rotary motion around its rotational axis until the first cam reaches the position indicated with broken lines. The inserted gradient coil unit 1 has thus been locked.

Although modifications and changes may be suggested by those skilled in the art, it is the intention of the inventors to embody within the patent warranted hereon all changes and modifications as reasonably and properly come within the scope of their contribution to the art.

We claim as our invention:

1. A magnetic resonance apparatus comprising:
a magnetic resonance scanner having an interior examination space;
a patient bed mechanism, adapted to receive an examination subject thereon, which is movable into and out of said examination space;
a gradient coil unit which is movable into and out of said examination space; and
a coupling unit for coupling said gradient coil unit to said patient bed mechanism for moving said gradient coil unit relative to said examination space with said patient bed mechanism, said coupling device automatically firmly joining said gradient coil unit to said patient bed mechanism when said patient bed mechanism moves toward and contacts said gradient coil unit in a movement direction, and releases said gradient coil unit from said patient bed mechanism when said patient bed mechanism again moves in said movement direction.

2. A magnetic resonance apparatus as claimed in claim 1 further comprising a cart for receiving said gradient coil unit outside of said examination space.

3. A magnetic resonance apparatus as claimed in claim 2 comprising a pivotable connection pivotably attaching said cart to said scanner adjacent to said examination space.

4. A magnetic resonance apparatus as claimed in claim 1 wherein said patient bed mechanism comprises electrical connections for supplying power to said gradient coil unit.

5. A magnetic resonance apparatus as claimed in claim 1 further comprising a guide device disposed in said examination space and interacting with said gradient coil unit in said examination space to guide said gradient coil unit through said examination space.

6. A magnetic resonance apparatus as claimed in claim 5 wherein at least a portion of said guide device also interacts with and guides said patient bed mechanism.

7. A magnetic resonance apparatus as claimed in claim 6 wherein at least a portion of said guide device extends into said patient bed mechanism.

8. A magnetic resonance apparatus as claimed in claim 1 wherein said coupling device is remotely actuatable to couple and uncouple said gradient coil unit and said patient bed mechanism.
